(19) United States
(12) Patent Application Publication
(10) Pub. No.: US 2008/0138327 A1
(43) Pub. Date: Jun. 12, 2008

(54) Substantially dry disposable device for creating ready-to-use solutions for cleaning and inhibiting the formation of biofilms on surfaces

(76) Inventor: Albert R. Kelly, Douglaston, NY (US)

Correspondence Address: EVELYN M. SOMMER, 570 LEXINGTON AVENUE , 17TH FLOOR, NEW YORK, NY, 10022, US

(21) Appl. No.: 12/069,383

(22) Filed: Feb. 11, 2008

Related U.S. Application Data
(63) Continuation-in-part of application No. 11/472,893, filed on Jun. 23, 2006.
(60) Provisional application No. 60/784,433, filed on Mar. 22, 2006.

(51) Int. Cl. A61K 38/43

(52) U.S. Cl. 424/94.1

(57) ABSTRACT

A substantially dry disposable device for creating ready-to-use solutions effective in cleaning and inhibiting the growth and formation of biofilms on surfaces and methods of manufacturing the same are disclosed. The device is impregnated with a concentrated formulation comprising a fermentation supernatant as obtained from the fermentation of Saccharomyces cerevisiae, at least one non-ionic surfactant, at least one fragrance, at least one dye and at lest one preservative. The composition used in the impregnation is a stable, fairly viscous, dark amber solution that can readily be impregnated into a non-woven, dried and conveniently packaged and stored for subsequent use. It can then be lifted or immersed into water to reconstitute an appropriate use-dilution.

This application is a continuation-in-part of application Ser. No. 11/472,893.

### FIELD OF THE INVENTION

The present invention generally relates to substantially dry disposable devices comprised of non-woven substrates formed from synthetic and/or cellulosic fibers impregnated with an ultra-concentrated bio-organic catalyst composition for cleaning and inhibiting the formation of biofilms on surfaces, and methods for using and for manufacturing the same. In particular, the present invention relates to a substantially dry disposable device comprised of a non-woven substrate formed from synthetic and/or cellulosic fibers impregnated with an ultra-concentrated bio-organic catalyst composition, comprising a fermentation supernatant, at least one non-ionic surfactant, at least one preservative, and optionally a natural essential oil sanitizer to provide the substantially dry devices. Further, the present invention relates to methods for using this substantially dry disposable device to reconstitute effective use-dilutions of the concentrate, by immersion of the substantially dry device in fixed amounts of water and using the reconstituted use-dilution for cleaning and inhibiting the growth and formation of biofilms on surfaces. In addition, the invention provides an ultra-concentrated bio-organic catalyst composition, comprising a fermentation supernatant, at least one non-ionic surfactant and at least one preservative, useful for cleaning and inhibiting the formation of biofilms on surfaces.

### BACKGROUND OF THE INVENTION

A biofilm is a community of microbes, embedded in an organic polymer matrix, adhering to a surface. In nutrient rich natural and industrial ecosystems, biofilm cells will predominate and cause problems as increased frictional resistance to fluids in water conduits, fouling on ship hulls, and decreased heat transfer from heat exchangers, corrosion of metallic substrata, and contamination in the food and biotechnology industry. Biofilms are also a severe problem in medical science and industry causing dental plaque, contaminated endoscope and contact lenses, prosthetic device colonization and biofilm formation on medical implants.

The biofilm matrix is a collection of micro-colonies with water channels in between and an assortment of cells and extracellular polymers (polysaccharides, glycoproteins, proteins). Pathogenic microbes growing in biofilms are more resistant to antibiotics and disinfectants than planktonic cells and the resistance increases with the age of the biofilm. Bacterial biofilm also exhibits increased physical resistance toward desiccation, and to extreme temperatures or light. As mentioned, biofilm formation causes industrial, environmental, and medical problems. The difficulties in cleaning, disinfection and inhibition in the formation of bacterial biofilm with chemicals is a major concern in many industries.

Many compositions and methods for degrading biofilms have been devised in the art. Harsh chemicals, elevated temperatures and vigorous abrasion procedures have been widely used. There are conditions, however, where these approaches cannot be used or are impractical e.g., caustic- and acid-sensitive environments, temperature or abrasion sensitive components that are associated with the biofilm.

The use of enzymes in degrading, removing and destroying biofilms is also known.

U.S. Pat. No. 6,830,745 teaches a two component composition comprising an anchor enzyme for degrading the biofilm structure and a second anchor enzyme for a bacteriostatic effect.

U.S. Pat. No. 5,411,666 teaches a composition for removing biofilm from industrial water systems, comprising at least two biological enzymes and a surface-active agent.

U.S. Pat. No. 6,100,080 teaches a method for treating biofilm using a cleaning composition comprising one or more hydrolases and a bactericidal disinfectant composition comprising laccase and an oxidation enhancer.

U.S. Pat. No. 6,777,223 teaches a method for eliminating the formation of biofilm using a composition comprising one or more acylases and a carrier to degrade a lactone produced by one or more microorganisms.

U.S. Pat. No. 6,699,391 teaches a method for reducing biofilm in an aqueous system using a mixture containing enzymes and surfactants.

Of particular relevance to the present invention are the compositions disclosed by Parker Dale in U.S. Pat. Nos. 5,820,758; 5,849,566; 5,879,928; 5,885,950 and PCT publication number WO97/27941. The compositions disclosed comprise a yeast fermentation supernatant, preservatives and a non-ionic surfactant and are indicated for the treatment of municipal and industrial wastewater, for cleaning grease-traps and septic tanks and for accelerating the decomposition of hydrocarbons. The compositions can also be used to degrade biofilms.

The compositions contain substantial amounts of water, the major component within the supernatant that is produced in the fermentation process. Because such aqueous compositions are particularly effective for inhibiting the growth and development of biofilms in aqueous environments, they have been supplied and dispensed from 1 gallon, 5 gallon, and 55 gallon containers, and even from 250-gallon totes. Bulk liquid containers like these require special handing to prevent spills and contamination, and incur substantial costs for shipping and storage. Because the aqueous compositions are all concentrates, they must be diluted at the point of use, in proportions appropriate for the specific applications involved. The use-dilution for most surface treatment applications is 1 part concentrate to 32 parts water.

The present invention offers a significant opportunity to facilitate, simplify and improve the safety and economics of delivery, handling and storage of these concentrates and to dispense the proper use-dilutions for cleaning surfaces and for breaking down and inhibiting the development of biofilms, in addition to the many other purposes to which these products lend themselves.

Significantly, the present invention provides an efficient and convenient system for making the bioorganic catalyst system available for a broad variety of surface cleaning applications in individual, institutional and home environments. The potential benefits for using this technology for a variety of surface cleaning applications accrues from its unique bioorganic catalyst mode of action that breaks down biofilms, prevents their reformation downstream and leaves no organic residues to support the re-growth of microbial colonies. For surface cleaning purposes, this technology provides a similar mode of action by precipitating a catalytic degradation of waste, including fats, oils and grease, leaving no organic residues to support the re-growth of microbial organisms.

The specific formulations of the present invention relate back to those described in U.S. Pat. No. 5,885,950, which in turn are similar to those described in U.S. Pat. No. 3,635,797, both of which patents are incorporated herein by reference. According to the disclosure of U.S. Pat. No. 5,885,950, yeast, Saccharomyces cerevisiae, is cultured in an aqueous medium comprising; a sugar source, such as sucrose from molasses or raw sugar, soy beans or mixtures thereof, a sugar concentration of about 10 to 30% by weight, is used; malt such as diastatic malt is used at a concentration of about 7 to 12% by weight; a salt, such as a magnesium salt, and in particular magnesium sulfate, is used at a concentration of about 1 to 3%, by weight, and yeast is added to the medium to a final concentration of about 1 to 5%, by weight.

The mixture is incubated at about 26 to 42° C. until the fermentation is completed, i.e., until effervescence of the mixture has ceased, usually about 2 to 5 days depending on the fermentation temperature. At the end of the fermentation, the yeast fermentation product is centrifuged to remove the “sludge” formed during the fermentation.

The supernatant (about 98.5%, by weight) is mixed with sodium benzoate (about 1%, by weight), imidazolidinyl urea (about 0.01%, by weight), diazolidinyl urea (about 0.15%, by weight), calcium chloride (about 0.25%, by weight) to form fermentation intermediate. The pH is adjusted to about 3.7 to about 4.2 with phosphoric acid. The composition of the fermentation intermediate is summarized in Table I.

TABLE I

| Fermentation | |
|---|---|
| Intermediate (I) Component | % by weight |
| Fermentation supernatant | 98.59 |
| Sodium benzoate | 1.00 |
| Imidazolidinyl urea | 0.01 |
| Diazolidinyl urea | 0.15 |
| Calcium chloride | 0.25 |
| Adjust pH | to about 3.7 to about 4.2 |
| | with phosphoric acid. |

The fermentation intermediate is prepared by filling a jacketed mixing kettle with the desired quantity of the fermentation supernatant. With moderate agitation, the pH is adjusted to 3.4 to 3.6 with phosphoric acid. With continuous agitation, sodium benzoate, diazolidinyl urea, imidazolidinyl urea, and calcium chloride are added. The temperature of the mixture is then slowly raised to about 40° C. and the mixture is agitated continuously. The temperature is maintained at about 40° C. for about one hour to ensure that all the components of the mixture are dissolved. The mixture is then cooled to about 20° C. to 25° C.

The fermentation intermediate is then formulated into the composition (II) to be used to form composition (III). Fermentation intermediate of about 12.31%, by weight, of the composition (II) is mixed with a nitrogen containing compound such as urea, ammonium nitrate or mixtures thereof (about 9%, by weight composition II), preservatives such as sodium benzoate (about 0.1%, by weight, of the final composition), imidazolidinyl urea (about 0.01%, by weight, of composition II), diazolidinyl urea (about 0.15%, by weight, of composition II) and mixtures thereof, a surfactant such as TERGITOL 15-S-7 (about 8%, by weight, of the composition II), triethanolamine (about 2%, by weight, of the composition II), and the composition is brought to 100% with water.

In the preferred embodiment, the composition comprises about 12.31% by weight fermentation intermediate, about 9% by weight of a nitrogen containing compound such as urea, ammonium nitrate or mixtures thereof, about 0.01% by weight imidazolidinyl urea, about 0.1% by weight sodium benzoate, about 0.15% by weight diazolidinyl urea, about 2% by weight triethanolamine, about 8% by weight of a surfactant such as Tergitol 15-S-7 and about 68.43% by weight of water (see Table II).

TABLE II

| Composition II Component | %, by weight |
|---|---|
| Ammonium nitrate | 9.00 |
| Tergitol 15-S-7 | 8.00 |
| Sodium Benzoate | 0.10 |
| Imidazolidinyl urea | 0.01 |
| Diazolidinyl urea | 0.15 |
| Triethanolamine | 2.00 |
| Fermentation Intermediate | 12.31 |
| Water | 68.43 |

The method for preparing composition II is to charge a mixing kettle with the desired volume of water at 20° C. to 25° C. The preservatives are added to the water with agitation. Tergitol 15-S-7 is then added and the mixture is blended until the solids are dissolved. Triethanolamine is then added and the mixture is again blended until the solids are dissolved. The fermentation intermediate is then added with gentle agitation. The pH is adjusted to about 8.5 to 9 with caustic soda. Water is added to complete the composition at 100%.

The final concentration of components in composition III is summarized in Table III.

TABLE III

| Composition III Component | %, by weight |
|---|---|
| Sodium Benzoate | 0.19 |
| Imidazolidinyl urea | 0.01 |
| Diazolidinyl urea | 0.15 |
| Tergitol 15-S-7 | 8.00 |
| Calcium Chloride | 0.03 |
| Triethanolamine | 2.00 |
| Fermentation Intermediate | 12.14 (clarified) |
| Ammonium nitrate | 9.00 |
| Water | 68.48 |
| Adjust pH | to about 8.5 to 9 with caustic soda. |

The composition III is then diluted or metered at differing dosages.

The aforesaid compositions III in appropriate dilutions have been used in breaking down biofilms in drains, grease traps, septic tanks and in breaking down organic pollutants in waste water treatments, and enhancement of water clarification and purification including the clarification of still bodies of water. The dilution for a particular purpose is based on effectiveness of the composition in meeting specific performance characteristics. Usually the final composition is diluted or metered to optimize the effectiveness in various environmental venues and to minimize costs.

The ultra-concentrate used to impregnate nonwoven substrates of this invention is distinct from the concentrates described above in U.S. Pat. No. 5,885,950. This is because the level of surfactants is increased, in part, to compensate for the absence of water added to that available in the fermentation supernatant. It was not expected that the omission of added water to this relatively unstable composition disclosed in those patents would be stable. Nor was it expected that such a composition could be reconstituted as an effective use-dilution, after being impregnated into a fabric, dried, and then immersed in finite amounts of water.

The ultra-concentrated composition of this invention is a slightly more viscous, homogeneous, and is an amber colored solution. After being impregnated into the substrate, and dried to remove any free water, the treated device is then processed and packaged for the desired end-use.

A key object of this invention is to provide a delivery system for this composition of the fermentation supernatant, obtained by culturing yeast Saccharomyces cerevisiae, which is easier to manufacture and to use.

Another object of the present invention is to provide a dry delivery system which can be immersed into variable, but finite, amounts of water to create ready-to-use solutions that will be effective in cleaning and breaking down biofilms on surfaces, and for other related applications.

A further object of the invention is to provide a disposable delivery device comprised of a non-woven substrate composed of synthetic and/or cellulosic fibers impregnated with this ultra-concentrate composition that is composed of a fermentation supernatant obtained by culturing yeast, Saccharomyces cerevisiae, at least one preservative, at least one surfactant, and optionally, a natural essential oil sanitizer, which has been dried after treatment to remove substantially all of the free water present within the substrate and which upon immersion in water will reconstitute those active ingredients into a stable and effective aqueous use-dilution.

Yet another object of the invention is to provide disposable substantially dry applicators impregnated with the composition in amounts that would be activated upon saturation with water to provide effective cleaning of surfaces and for breaking down any biofilms that may be present.

Still, a further object of the invention is to provide disposable substantially dry delivery devices in the form of sleeves for spray bottle dip-tubes, wipes, pads, or applicators which have been impregnated with the concentrate composition comprised of a fermentation supernatant obtained by culturing yeast; Saccharomyces cerevisiae, at least one preservative, at least one surfactant, and optionally, a natural essential oil sanitizer, for use in creating use-dilutions that are effective for cleaning and breaking down biofilms on surfaces.

Still another object of the invention is to provide an ultra-concentrate of the fermentation supernatant comprised of enzymes from a Saccharomyces cerevisiae fermentation culture, which can then be used to impregnate non-woven fabrics to produce a substantially dry device, which can then be used to clean surfaces, and to break down biofilms.

These and other objects of the invention will become apparent from the following specification and claims.

### SUMMARY OF THE INVENTION

The present invention is directed to a substantially dry disposable delivery device for creating ready-to-use solutions for cleaning and breaking down biofilms on surfaces. The device comprises a substantially dry non-woven substrate formed from synthetic and/or cellulosic fibers that is impregnated with an ultra-concentrate composition of this invention that comprises a yeast fermentation supernatant to which no additional water has been added, at least one preservative and at least one surfactant. The concentrate may further comprise an essential oil sanitizing compound, at least one fragrance, and at least one dye to enhance the appearance and other properties of the use-dilution.

Preferred embodiments of the non-woven device are in the form of an impregnated sleeve for a spray bottle dip-tube, an applicator, a wipe, a pad, a towel or the like. The devices can be manufactured by impregnating non-woven substrates with the concentrate, further drying of the impregnated non-woven substrates and forming them into the desired form needed to create active use-dilutions in water, and then packaging the same. The ready-to-use dilution is created when the impregnated device is immersed in finite amounts of water.

### DETAILED DESCRIPTION OF THE INVENTION

The present invention provides a disposable device impregnated with an ultra-concentrate comprising a fermentation supernatant, at least one preservative, and at least one surfactant to which no additional water has been added, and which can be used conveniently, for re-creating a ready-to-use dilution effective for cleaning and breaking down biofilms on surfaces. Selected fragrances and dyes can also be included in the concentrate to provide ancillary benefits if desired. The ultra concentrate is produced by adding no additional water to the supernatant made in accordance with procedures similar to those disclosed in U.S. Pat. No. 5,885,950 and PCT publication number WO97/27941.

The following describes the ultra-concentrate composition of the invention used for impregnation into nonwoven delivery devices. The stability, versatility, and clarity of this ultra concentrate fermentation supernatant composition was not to be expected.

The ultra-concentrate is an aqueous solution comprising preservatives, non-ionic and anionic surfactants, and fermentation supernatant from Saccharomyces cerevisiae.

Using the original concentrate composition III of (U.S. Pat. No. 5,855,950), it was determined that a 1:32 dilution was ideal for hard surface cleaning, deodorizing and for breaking down and inhibiting the development of biofilms.

This dilution was prepared by introducing the original concentrate (U.S. Pat. No. 5,855,950) in a typical one quart spray bottle and requires that (1) 28 grams be added to 896 grams of water. If that amount of concentrate were to be added to a nonwoven strip or sleeve weighing 2.5 grams to 2.84 grams, it would represent an add-on of greater than a 1,000%, and would not be retainable within the nonwoven sleeve.

Using a sleeve weight of a trilaminate material measuring 2.25″×8.5″ (see examples), effective use-dilutions in 896 grams of water were prepared with active ingredient add-ons of the ultra concentrate of this invention in amounts of 9.5 grams (1:94) and 7.11 grams (1:126). Impregnations of these amounts in sleeves weighing 2.5 grams and 2.84 grams respectively equaled add-ons of 380% and 250% and were retainable.

Because both sleeve weights and use-dilution volumes can be varied and are independent, the specifications that differentiate the ultra concentrate of this invention from the original concentrate disclosed example in U.S. Pat. No. 5,855,950 are the significantly lower active ingredient concentrations required in use-dilutions to deliver comparable cleaning effectiveness. The previously available concentrate at a dilution ratio of 1:32 represents an active concentration of 3.1%. The ultra concentrate of the invention in use-dilution ratios of 1:94 and 1:126 represent active ingredient concentrations of 1.1% and 0.8% respectively. These lower active ingredient concentrations of the ultra concentrates are highly effective and reduce impregnation loads in the sleeves to levels which are readily retainable.

The preferred active ingredient concentration is from 0.2% to 2.0%. This is considerably lower than that required with the original concentrates, which required a minimum active ingredient concentration of 3.0%, which is 50% higher than high end of the range in accordance with the invention. Preferably the total addons of the concentrate represent 200-400% of the substrates' total weight.

A preferred composition comprises the following:

| | | |
|---|---|---|
| 1) Fermentation Supernatant; | 53.9% | by weight |
| 2) Tergitol 15-S-7, non-ionic surfactant: | 39.5% | by weight |
| 3) Dow fax, anionic surfactant: | 4.4% | by weight |
| 4) Germaben 2: | 1.3% | by weight |
| 5) Sodium Benzoate: | 0.9% | by weight |
| 6) Germall 115: | 0.0008% | by weight |

The foregoing composition has a substantially greater total surfactant content (43.9%), and in this embodiment combines anionic surfactants with the non-ionic surfactant to provide enhanced grease cleaning attributes, which function in concert with the catalytic breakdown of organic contaminates by the fermentation supernatant.

When this composition is diffused into water, the fermentation supernatant combines with the surfactants to form activated microbubbles. These functionalized microbubbles provide enhanced gas transfer across lipid molecular structures, resulting in the cleaving of ester bonds in fats, oils, and grease, while providing a vastly accelerated natural catalytic bio-oxidation of the molecular elements. This natural catalytic bio-oxidation is effective in air, water, and on hard surfaces.

Non-ionic surfactants suitable for use in the present invention include, but are not limited to, polyether non-ionic surfactants comprising fatty alcohols, alkyl phenols, fatty acids and fatty amines which have been ethoxylated; polyhydroxyl non-ionic (polyols) typically comprising sucrose esters, sorbital esters, alkyl glucosides and polyglycerol esters which may or may not be ethoxylated.

As replacements for the foregoing nonionic surfactants, bio-based nonionic surfactants such as Sugafax D-12 and Colalap M-259, both from Colonial Chemical Inc. and Cocamide DEA derived from coconut oil may also be used.

Anionic surfactants suitable for use in the present invention include, but are not limited to, those selected from the group consisting of alkyl and alkyl ether sulfates, sulfated monoglycerides, sulfonated olefins, alkyl aryl sulfonates, primary or secondary alkane sulfonates, alkyl sulfosuccinates, acyl taurates, acyl isethionates, alkyl glycerylether sulfonate, sulfonated methyl esters, sulfonated fatty acids, alkyl phosphates, ethoxylated alkyl phosphates, acyl glutamates, acyl sarcosinates, alkyl sulfoacetates, acylated peptides, alkyl ether carboxylates, acyl lactylates, anionic fluorosurfactants, and combinations thereof. Combinations of anionic surfactants can be used effectively in the present invention. Specific examples of alkyl sulfates that may be used are sodium, ammonium, potassium, magnesium, or TEA salts of lauryl or myristyl sulfate. Examples of alkyl ether sulfates that may be used include ammonium, sodium, magnesium, or TEA laureth-3 sulfate.

Preservatives suitable for use in the present invention include, but are not limited to, sodium benzoate, imidazolidinyl urea, diazolidinyl urea and mixtures thereof The formulation identified above with significantly higher surfactant content is the preferred composition with no additional water having been added to the concentrate. The result is that the active ingredients, including the fermentation supernatant, non-ionic, and anionic surfactants and preservatives represent 100% of the total composition with no water added. Additionally, selected natural fragrances such as eucalyptus can be added along with one or more dyes to enhance the odor and appearance of the use-dilution. Fragrances are included in the amount of 0.001 to 3% by weight. Dyes, most preferably green dyes, can be included in the amount of about 0.003% by weight.

The preferred substrate should preferably incorporate any highly absorbent non-woven materials that are comprised of a plurality of cellulosic fibers. A preferred substrate for the sleeve is a composite material supplied by Texel, comprising 65% Tencel and 35% polypropylene in basis weights of 136, 203, 271 and 339 grams per square meter. Alternatively, a sonically bonded trilaminate substrate at a basis weight of about 203 grams per square meter. per square yard supplied by Tudor Converted Products comprising either a 100% cellulosic wadding core supplied by Shawano Paper, a needlepunch Tencel core supplied by Texel or a Spunlaced Tencel core supplied by Ahlstrom Green Bay, LLC. These substrates can be converted into any of the following formats, including single-layer strips, multi-layer composites, or as sleevelets if the use-dilutions is to be reconstituted in a spray bottle, as for example, described in U.S. Pat. No. 6,250,511, the entirety of which is incorporated herein by reference thereto. A larger device such as a wipe, towel or pad is preferred if the solution is to be reconstituted in a larger container, such as a mop bucket, or a water tight, heavy-duty bag.

During manufacture, the substrate is first, impregnated with the desired amount of the ultra concentrate formed into the desired device, and then packaged within a sanitary film wrap. This packaging is designed to allow substantially all the free water present to evaporate, but protect the device from any contamination. Free water is removed by the drying, resulting in a substantially dry impregnated device.

In use, the impregnated device is immersed in finite amounts of water. The active compositions impregnated in the device are gradually released into water, forming a ready-to-use dilution. Gentle shaking of the solution can expedite release of the active compositions. The concentration of the final solution can be controlled by adjusting the amounts of water used, or the concentrate load per device.

The device is to be supplied in an impregnated condition, and properly packaged in a breathable and sanitary enclosure. In use, the user opens the enclosure, removes the device and immerses it into a container of water, such as a spray bottle, a bucket, a bag, and the like, each having been provided with the correct amount of water needed to reconstitute the ready-to-use dilution. The device can be supplied within a water-impermeable bag, which serves both as packaging for the device, and a container for preparing the use-dilution.

For example, the device can be fixed to the interior wall of a bag. In use, the user opens the bag, adds the desired amount of water to reconstitute the ready-to-use dilution. Preferably, the bags are of the type that can stand on its bottom by itself, such as those frequently used in the food packaging industry for drinks, etc., and the ones disclosed in U.S. Pat. Nos. 6,659,645; 6,783,277; 5,788,378; and 4,954,124.

The ultra concentrate is stable and can be stored in appropriate containers until the time required for further processing.

### EXAMPLES

1. Ultra-Concentrate Reduced-Water Composition

The ultra-concentrate formulation of the composition disclosed herein is used to produce the device of this invention. Using substantially the same process as disclosed in U.S. Pat. No. 5,855,950, the ultra-concentrate composition is however formulated by adding significant amounts of surfactants despite the absence of added water to that present in the used fermentation supernatant. The result is a more viscous and dark amber solution that is more homogeneous and exhibits surprising clarity.

In order to confirm the water content, laboratory tests were performed by a procedure in which six 2.5″×8.5″ strips, made of a 203 grams per square meter. trilaminate polypropylene substrate, weighing 2.5 grams each were impregnated with an average of 9.5 grams of the afore-described ultra-concentrate fermentation, coded (4BZOC-5BO7W). These strips were then placed on racks in a ventilated oven at 130° F. so as to evaporate all free water. Upon completion of the drying process, the resultant weights confirmed that the bound-water represents 54.45% of the composition's original weight. Total “active” ingredients, excluding the bound water present in the fermentation supernatant, represented 45.55% of the 9.4 grams impregnated, or 4.375 grams.

This is a substantial reduction of the water content to that which was present in Composition III (U.S. Pat. No. 5,855,950) and which was determined by the same method to be 88.4%. Thus, this ultra-concentrate, reduced water composition lowered the presence of water by 38.4%, and more significantly, increased the level of “active” ingredients from 11.59% to 45.55%, representing nearly a four-fold increase of +393%.

2. Treated Dip Tube Sleeve for Creating an Effective Multi-Puriose Cleaning Solution in a 32 oz. Spray Bottle.

Using Composition III (U.S. Pat. No. 5,855,950), it was determined that a 1:32 dilution was the ideal blend for the hard surface cleaning, deodorizing and for breaking down and inhibiting the development of biofilms. Because the ultra-concentrate formulation of this invention contains a 393% increase in the level of active ingredients, the use dilution needed with the new formulation to replicate the same performance is reduced by that amount, to 1 part ultra concentrate in 126 parts water.

A 32 oz. spray bottle containing 896 grams of fluid, when divided by 126 equals 7.05 grams. That amount of the new ultra concentrate of this invention was impregnated into trilaminate nonwoven material measuring 2.25″×8.5″, which weighed 2.84 grams. This trilaminate material was comprised of a needlepunch Tencel core at a basis weight of 169 grams per square meter and outer layers of 34 grams per square meter spunbonded and/or thermal bonded polypropylene, all three layers of which were laminated ultra-sonically. This material was formed into a sleevelet, coated with 7.05 grams of the new concentrate, and slipped onto the diptube of the sprayer mechanism of a 32 oz. spray bottle.

The spray bottle was filled with tap water at room temperature. The diptube with the treated sleeve secured by an especially designed retainer at the bottom end was then inserted into the bottle and the screw cap secured. The bottle was then shaken to facilitate rapid release of the actives into solution. When the treated sleeve was observed to have become white, the properly formulated use-dilution was ready for use.

This use-dilution was then evaluated for efficacy in a variety of surface cleaning applications. For basic counter top and tabletop cleaning, it cleans as well or better than the leading selling brands of multi-surface cleaners, and could be sprayed safely on virtually all surfaces including cutting boards, utensils, and appliances.

Broilers and grills used to cook chicken were sprayed liberally with this use-dilution, after use. They were allowed to stand after treatment for up to 5 minutes, after which an abrasive cloth, brush, and/or scraper was used with warm water to quickly and easily break down and remove all of the baked-on debris and to dissolve oil and greasy residues. Compared to soaking such items in a typical detergent based solution, this new method was significantly faster and more efficient.

Stovetops and microwaves collect baked-on oils and debris which are difficult to break down and remove. When sprayed liberally with this new use-dilution, activated micro-bubbles were observed to form and to visibly begin to rupture and break apart such debris. Within approximately 60 to 120 seconds, these surfaces were wiped clean without rinsing or additional water being added.

Cutting boards and utensils used to prepare and cook fish products of all types are difficult to clean and especially to deodorize, after use. When this new use-dilution was simply sprayed on these surfaces the fishy odor was quickly neutralized. With the benefit of mild rinsing, all of the oils were dissolved and the baked-on residue was easily wiped away.

We claim:

1. A substantially dry impregnated disposable device for creating ready-to-use dilutions effective for cleaning and inhibiting the formation and growth of biofilms on surfaces, comprising a water-insoluble absorbent substrate, having incorporated therein an amount of about 200-400% of the substrate's total basis weight of an ultra concentrate composition, comprising at least one non-ionic surfactant, at least one preservative, and a fermentation supernatant comprising active enzymes from a Saccharomyces cerevisiae fermentation culture, wherein effective use dilution ratios of the ultra concentrate composition in diluent amount to about 0.2 to 2.0%.

2. A substantially dry impregnated disposable device according to claim 1 having incorporated therein 250-380% of the substrate's total basis weight of said ultra concentrate composition.

3. A substantially dry impregnated disposable device according to claim 1 wherein said substrate comprises a member selected from the group consisting of synthetic fibers, cellulosic fibers and mixtures thereof.

4. A substantially dry impregnated disposable device according to claim 1 wherein said non-ionic surfactant is selected from the group consisting of polyether non-ionic surfactants comprising ethoxylated fatty alcohols, alkyl phenols, fatty acids and fatty amines and ethoxylated and non-ethoxylated polyhydroxyl non-ionic surfactants comprising sucrose esters, sorbital esters, alkyl glucosides, polyglycerol esters and bio-based nonionic surfactants selected from the group consisting of Sugafax D-12, Colalap M-259, and Cocamide DEA.

5. A substantially dry impregnated disposable device according to claim 1 wherein said preservative is selected from the group consisting of sodium benzoate, imidazolidinyl urea, diazolidinyl urea, and mixtures thereof.

6. A substantially dry impregnated disposable device according to claim 1 wherein said ultra concentrate composition further comprises at least one dye.

7. A substantially dry impregnated disposable device according to claim 1 wherein said ultra concentrate composition further comprises at least one fragrance.

8. A substantially dry impregnated disposable device according to claim 1 wherein said ultra concentrate composition additionally contains at least one anionic surfactant.

9. A substantially dry impregnated disposable device according to claim 1 wherein said ultra concentrate composition additionally contains at least one natural essential oil sanitizer.

10. A substantially dry impregnated disposable device according to claim 1 wherein said ultra concentrate composition contains a fermentation supernatant from Saccharomyces cerevisiae.

11. A substantially dry impregnated disposable device according to claim 1 wherein effective use-dilution ratios of the ultra concentrate composition in diluent amount to from about 0.2 to about 2.0%.

12. A substantially dry impregnated disposable device according to claim 1 wherein effective use-dilution ratios of the ultra concentrate composition in diluent amount to about 0.8 to about 1.1%.

13. A substantially dry impregnated disposable device according to claim 1 wherein said ultra concentrate composition comprises a fermentation supernatant at 53.9 wt %, Tergitol 15-S-7 as a non-ionic surfactant at 39.5 wt %, Dow fax as an anionic surfactant at 4.4 wt %, Germaben 2 at 1.3 wt %, Sodium Benzoate at 0.9 wt % and Germall 115 at 0.0008 wt %.

14. A substantially dry impregnated disposable device according to claim 1 wherein said ultra concentrate composition is present in amounts required to yield an appropriate use-dilution for cleaning various surfaces when said device is saturated with water.

15. A substantially dry disposable device impregnated according to claim 1, adapted to be placed within the water-filtering vessel for a pool or large body of standing water to release a dilution of the composition sufficient to aid in the clarification and purification of the water, and to inhibit the formation of biofilms on interior surfaces.

16. A substantially dry disposable device impregnated according to claim 1, shaped as a spray bottle sleeve and adapted to be placed onto the diptube of a spray bottle, said spray bottle filled with water before use and said spray bottle sleeve being immersed in said water when said diptube is inserted into said spray bottle to yield a dilution of the ultra concentrate composition required for the cleaning of hard surfaces and to inhibit the formation of biofilms on said hard surfaces when said dilution is sprayed onto said hard surfaces.

17. A substantially dry disposable device impregnated according to claim 1, shaped as a scrubbing pad and adapted to be immersed or dipped into water to reconstitute an appropriate use-dilution of the ultra concentrate composition, incorporated in said pad, sufficient to aid in the cleaning of the hard surfaces and to inhibit the formation of biofilms on said hard surfaces.

18. A system for creating ready-to-use solutions capable of cleaning or inhibiting the formation and growth of biofilms on surfaces, upon the addition of water, comprising a substantially dry impregnated disposable device according to claim 1 and a water impermeable bag, wherein said device is fixed onto the inner wall of said bag.

19. A treatment composition for cleaning and inhibiting the formation and growth of biofilms on surfaces comprising, at least one non-ionic surfactant, at least one fragrance, at least one dye, at least one preservative, and concentrated fermentation supernatant comprising active enzymes directly as produced in a Saccharomyces cerevisiae fermentation, as active ingredient wherein effective use dilution ratios of said concentrated fermentation supernatant in diluent amount to about 0.2 to 2.0%.

20. A treatment composition for cleaning and inhibiting the formation and growth of biofilms on surfaces according to claim 19 wherein said composition further comprises at least one anionic surfactant.

21. A treatment composition for cleaning and inhibiting the formation and growth of biofilms on surfaces according to claim 19 wherein said composition further comprises at least one natural essential oil sanitizer.

22. A method of manufacturing the substantially dry disposable device according to claim 1, comprising the steps of,
a. impregnating said substrate with specific amounts of the treatment composition according to claim 19, and
b. drying said substrate to remove substantially all free water.
